(19) United States
(12) Patent Application Publication
(10) Pub. No.: US 2015/0313896 A1
(43) Pub. Date: Nov. 5, 2015

(54) METHOD FOR TREATING CELL PROLIFERATION DISORDERS

(71) Applicant: Galderma Research & Development, Biot (FR)

(72) Inventors: Guy BOUVIER, Biot (FR); Carine ROSIGNOLI, Mougins (FR); Christelle NONNE, Valbonne (FR); Emmanuel VIAL, Nice (FR)

(21) Appl. No.: 14/799,026

(22) Filed: Jul. 14, 2015

Related U.S. Application Data
(63) Continuation-in-part of application No. PCT/US14/48439, filed on Jul. 28, 2014.
(60) Provisional application No. 62/096,233, filed on Dec. 23, 2014.
(60) Provisional application No. 61/858,885, filed on Jul. 26, 2013.

(51) Int. Cl. A61K 31/498; A61K 9/12; A61K 9/06; A61K 45/06; A61K 9/00

(52) U.S. Cl. CPC A61K 31/498; A61K 45/06; A61K 9/0014; A61K 9/06; A61K 9/122

(57) ABSTRACT

Methods, compositions and products for treating or reducing a cell proliferation disorder, such as hand and foot syndrome or cutaneous T cell lymphoma, in a subject in need thereof are described. The methods involve topically administering to the subject a composition containing an α adrenergic receptor agonist, such as brimonidine.

### CROSS REFERENCE TO RELATED APPLICATIONS

This application is a Continuation-in-part of International Application No. PCT/US14/48439, filed Jul. 28, 2014, which was published in the English language on Jan. 29, 2015, under International Publication No. WO2015/013709, which is entitled to priority pursuant to 35 U.S.C. §119(e) to U.S. Provisional Patent Application No. 61/858,885, filed Jul. 26, 2013, and claims priority from U.S. Provisional Patent Application No. 62/096,233, filed Dec. 23, 2014, the disclosure of which is hereby incorporated by reference herein in its entirety.

### BACKGROUND OF THE INVENTION

Abnormal cell proliferation can be triggered by various causes, including but not limited to, UV-radiation and cancer therapy. Epidermal Growth Factor Receptor, or EGFR (also referred to as ErbB1 and HER1), is a cell surface protein, which is activated by binding of its specific ligands, such as epidermal growth factor and transforming growth factor α (TGFα), etc. The activation of EGFR causes cell proliferation, inhibition of cell death, and increased epidermal hyperplasia, i.e., the increase in number and size of normal cells in normal arrangement.

When skin is stimulated by UV radiation, EGFR increases keratinocyte proliferation, suppresses apoptosis, and augments and accelerates epidermal hyperplasia. (T. B. El-Abaseri and L. A. Hansen, “EGFR Activation and Ultraviolet Light Induced Skin Carcinogenesis,” Journal of Biomedicine and Biotechnology, vol. 2007, Article ID 97939, 4 pages, 2007). When stimulated by UV radiation, keratinocyte is activated through an EGFR dependent process and begins to create an abundance of keratinocyte cells. When the skin is exposed to UV-irradiation, apoptosis is suppressed by the EGFR-dependent pathway, causing cells to live longer than usual and also to carry genetic mutations that may lead to disease formation. The suppression of apoptosis, along with keratinocyte proliferation, considerably increases skin thickness and risks of other cell proliferation disorders or conditions.

Epidermal hyperplasia can be partially attributed to an injury in the basal layer keratinocytes of the cell. Shortly after skin is exposed to UV radiation the keratinocytes begin to divide and multiply at more rapid pace. The EGFR-dependent process can trigger the increase in cell production in any type of cells, from regular cells, cells suffering from epidermal hyperplasia, to the increased production of cancer cells. Pharmacological inhibition of the UV-induced activation of EGFR in genetically initiated mouse skin tumorigenesis model suppresses tumorignesis and the activation of mitrogen-activated protein (MAP) kinases and phosphatidyl inositol-3-kinase (PI3K)/AKT signaling pathways. (T. B. El-Abaseri and L. A. Hansen, 2007, supra).

When a subject is treated with certain cancer therapies, especially certain chemotherapy drugs, the therapies may affect the growth of skin cells or capillaries (small blood vessels) in the hands and feet, which may result in hand and foot syndrome (HFS) characterized by dysesthasia and erythema on the hands and feet several days after treatment. In some extreme cases, these symptoms may evolve into blistering desquamation, crusting, ulceration, and epidermal necrosis. Biopsies show marked hyperkeratosis with parakeratosis in the stratum corneum of the epidermis, spongiosis with numerous pyknotic cells without associated lymphocytes in the stratum malpighii, focal areas of vacuolization in the basal layer, and a mild perivascular lymphocytic infiltrate and melanin deposition in the dermis. (D. Lorusso et al., Pegylated liposomal doxorubicin-related palmar-plantar erythrodysesthesia (‘hand-foot’ syndrome). Ann Oncol (2007) doi: 10.1093/annonc/md1477). However, underlying causes for HFS are largely unknown.

The α adrenoceptor agonists have been used therapeutically for a number of conditions including hypertension, congestive heart failure, angina pectoris, spasticity, glaucoma, diarrhea, and for the suppression of opiate withdrawal symptoms (J. P. Heible and R. R. Ruffolo Therapeutic Applications of Agents Interacting with α-Adrenoceptors, p.180-206 in Progress in Basic and Clinical Pharmacology Vol. 8, P. Lomax and E. S. Vesell Ed., Karger, 1991). Published US Patent Application US20050276830 discloses α2 adrenergic receptor agonists and their use for treating or preventing inflammatory skin disorders.

There is a need of methods and compositions that would effectively treat or inhibit the progression of cell proliferation disorder associated with various maladies. The present invention relates to such improved methods and compositions.

### BRIEF SUMMARY OF THE INVENTION

Treatment with an α adrenergic receptor agonist, such as brimonidine, has resulted in a significant reduction of cell proliferation disorder in mammals, such as mice exposed to UV radiation.

In one general aspect, embodiments of the present invention relate to a method of treating a cell proliferation disorder in a subject in need thereof, comprising topically administering to a skin area of the subject a topical composition comprising an effective amount of at least one alpha adrenergic receptor agonist and a pharmaceutically acceptable carrier, wherein the skin area has, or is prone to have, the cell proliferation disorder.

In one embodiment, the cell proliferation disorder is induced by an UV-irradiation, such as by sun exposure.

In another embodiment, the cell proliferation disorder is associated with chemotherapy. Preferably, the cell proliferation disorder is a hand and foot syndrome or a cutaneous T cell lymphoma. More preferably, the alpha adrenergic receptor agonist is brimonidine.

Other aspects, features and advantages of the invention will be apparent from the following disclosure, including the detailed description of the invention and its preferred embodiments and the appended claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, the drawings show embodiments which are presently preferred. It should be understood, however, that the invention is not limited to the precise arrangements and instrumentalities shown.

In the drawings:

FIG. 1 shows various hematoxylin and eosin (H&E) stained mouse dorsal skin sections from UVB-irradiated or non-irradiated mice treated with vehicle or 2% (w/w) brimonidine tartrate, or as controls, treated with ethanol and water (EtOH/H₂O) or 4% (w/w) EGFR inhibitor;

FIG. 2 is a quantification of the epidermis thickness of mouse skin samples after receiving different treatments;

FIG. 3 shows various 5-ethynyl-2′-deoxyuridine (EdU) and 4′,6-diamidino-2-phenylindole (DAPI) stained mouse skin samples from UVB-irradiated and non-irradiated mice;

FIG. 4 is a quantification of the EdU stained cells of mouse skin samples after receiving different treatments; and

FIG. 5 is a photograph of H&E stained human skin showing the presence of alpha2α adrenergic receptors in vessels and sweat glands.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

As used herein, an “α adrenergic receptor agonist” or “agonist of α adrenoceptor” means a compound that binds to and stimulates alpha adrenergic receptor. An “α adrenergic receptor agonist” can be selective for an α1 adrenergic receptor, selective for an α2 adrenergic receptor, or nonselective for both an α1 adrenergic receptor and an α2 adrenergic receptor.

As used herein, the name of a compound is intended to encompass all possible existing isomeric forms (e.g., optical isomer, enantiomer, diastereomer, racemate or racemic mixture), esters, prodrugs, metabolite forms, or pharmaceutically acceptable salts, of the compound. For example, “brimonidine” can be the compound (5-bromo-quinoxalin-6-yl)-(4,5-dihydro-1H-imidazol-2-yl)-amine, and any pharmaceutically acceptable salt of the compound, such as brimonidine tartrate.

In an embodiment of the present invention, the α adrenergic receptor agonists include, but are not limited to, the α adrenergic receptor agonists disclosed in the published US Patent Application US20050276830, which is herein incorporated by reference in its entirety.

Representative α adrenergic receptor agonists that can be used in the present invention include, but are not limited to, those listed in Table 1.

TABLE 1
Representative α adrenergic receptor agonists

| Compound Formula | Compound Name |
|---|---|
| 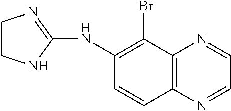 [SMILES: Brc1c(NC2=NCCN2)ccc2nccnc12] | (5-Bromo-quinoxalin- 6-yl)-(4,5-dihydro- 1H-imidazol- 2-yl)-amine (Brimonidine) |
| 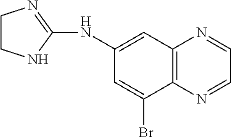 [SMILES: Brc1cc(NC2=NCCN2)cc2nccnc12] | (8-Bromo-quinoxalin- 6-yl)-(4,5-dihydro- 1H-imidazol-2- yl)-amine |
| 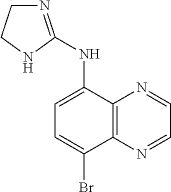 [SMILES: Brc1ccc(NC2=NCCN2)c2nccnc12] | (8-Bromo-quinoxalin- 5-yl)-(4,5-dihydro- 1H-imidazol-2- yl)-amine |
| 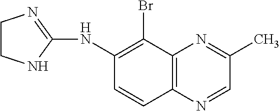 [SMILES: Cc1cnc2ccc(NC3=NCCN3)c(Br)c2n1] | (5-Bromo-3-methyl- quinoxalin-6-yl)- (4,5-dihydro-1H- imidazol-2- yl)-amine |
| 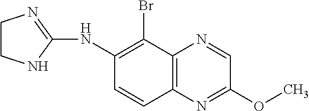 [SMILES: COc1cnc2c(Br)c(NC3=NCCN3)ccc2n1] | (5-Bromo-2-methoxy- quinoxalin-6-yl)- (4,5-dihydro-1H- imidazol-2-yl)-amine |
| 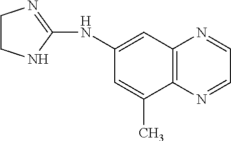 [SMILES: Cc1cc(NC2=NCCN2)cc2nccnc12] | (4,5-dihydro-1H- imidazol-2-yl)-(8- methyl-quinoxalin- 6-yl)-amine |
| 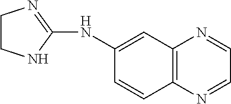 [SMILES: c1cnc2cc(NC3=NCCN3)ccc2n1] | (4,5-dihydro-1H- imidazol-2-yl)- quinoxalin-5- yl-amine |
| 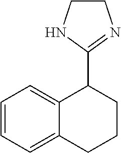 [SMILES: c1ccc2c(c1)CCCC2C1=NCCN1] | Tetrahydrozaline |
| 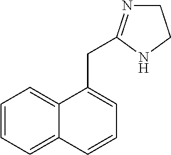 [SMILES: c1ccc2c(CC3=NCCN3)cccc2c1] | Naphazoline |
| 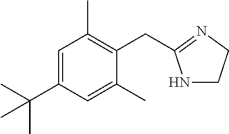 [SMILES: Cc1cc(C(C)(C)C)cc(C)c1CC1=NCCN1] | Xylometazoline |
| 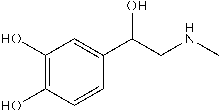 | Epinephrine |
| 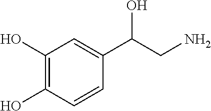 [SMILES: NCC(O)c1ccc(O)c(O)c1] | Norepinephrine |
| 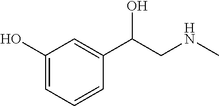 | Phenylephrine |
| 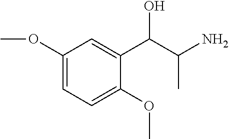 | Methoxyamine |

Preferably, the α adrenergic receptor agonist is an α2 adrenergic receptor agonist, most preferably brimonidine, (5-Bromo-quinoxalin-6-yl)-(4,5-dihydro-1H-imidazol-2-yl)-amine and pharmaceutically acceptable salts thereof, such as the tartrate salt of brimonidine.

Other examples of α adrenergic receptor agonists that can be used in the present invention include, but are not limited to, Dexmedetomidine, Medetomidine, Romifidine, Clonidine, Detomidine, Lofexidine, Xylazine, Tizanidine, Guanfacine, and Amitraz.

The phrase “pharmaceutically acceptable salt(s),” as used herein, means those salts of a compound of interest that are safe and effective for topical use in mammals and that possess the desired biological activity, Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds used in the present invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977), incorporated herein by reference.

As used herein, the term “hydrate” means a compound of interest, or a pharmaceutically acceptable salt thereof that further includes a stoichiometric or non-stoichiometric amount of water bound to it by non-covalent intermolecular forces.

As used herein, the term “subject” means any mammal, preferably a human, to whom will be or has been administered compounds or formulations according to embodiments of the invention.

As used herein, the term “composition” is intended to encompass a product comprising the specified ingredients in the specified amounts, as well as any product which results, directly or indirectly, from combinations of the specified ingredients in the specified amounts.

As used herein, a “pharmaceutically-acceptable carrier” means a carrier that is pharmaceutically or cosmetically suitable for use in the present invention without causing undue or unacceptable toxicity, incompatibility, instability, irritation, allergic response, and the like. This term is not intended to limit the ingredient which it describes.

One general aspect of the present invention relates to a method of treating a cell proliferation disorder in a subject in need thereof. The method comprises topically administering to a skin area of the subject a topical composition comprising an effective amount of at least one alpha adrenergic receptor agonist and a pharmaceutically acceptable carrier, wherein the skin area has, or is prone to have, the cell proliferation disorder.

Another general aspect of the present invention relates to a method of inhibiting or preventing a cell proliferation disorder induced by an UV-irradiation in a subject in need thereof, comprising topically administering to a skin area of the subject a topical composition comprising an effective amount of at least one alpha adrenergic receptor agonist and a pharmaceutically acceptable carrier.

As used herein, “a cell proliferation disorder induced by an UV-irradiation” refers to a cell proliferation disorder that occurs or develops resulting from the exposure of the skin to a UV radiation, excluding such skin disorder of a different etiology. Any skin disorder induced by UV-irradiation, including but not limited to, low grade, e.g., grade 1, of erythema or flaking, wrinkling or white raised area on skin, can be inhibited or reduced by the present invention.

In one embodiment of the present invention, the topical composition is administered to the skin area before the UV-irradiation.

In another embodiment of the present invention, the topical composition is administered to the skin area after the UV-irradiation.

In yet another embodiment of the present invention, the topical composition is administered to the skin area before and after the UV-irradiation.

As used herein, “topical application,” “topical administration” or “topically applying” means direct application or administration onto skin or any other epithelium in need of treatment. According to embodiments of the present invention, a topical composition can be topically administered by directly laying or spreading on the skin or epithelium in need of the treatment, e.g., by use of the hands, an applicator or any other means.

As used herein, a “cell proliferation disorder” includes any abnormal increase or decrease in the number of a cell in a tissue. The cell proliferative disorder can be induced, for example, by UV radiation or chemotherapy, either directly or indirectly. A “cell proliferation disorder” can be an abnormal increase in the number and/or volume of a cell or tissue in any layer of the skin, e.g., in the epidermis, dermis, and hypodermis. The cell or tissue in the skin can be, for example, keratinocytes, Merkel cells, melanocytes, Langerhans cells, fat cells, connective tissue, etc.

According to embodiments of the present invention, the “cell proliferation disorder” can be associated with one or more conditions, such as that selected from the group consisting of sun exposure, hormonal imbalance, a deficiency in vitamin and/or antioxidant, epidermal hyperplasia, keratinocyte proliferation, EGFR-dependent cell division, chemotherapy-induced hand and foot syndrome, cutaneous T cell lymphoma, and combinations thereof.

According to some embodiments of the present invention, the “cell proliferation disorder” is not associated with a skin tumor, which includes a skin cancer, a benign skin tumor and pre-malignant skin tumor, nor is “cell proliferation disorder” associated with rosacea, telangiectasias psoriasis, purpura, sagging skin or wrinkle.

As used herein, the term “cell proliferation disorder” encompasses epithelium hyperplasia, proliferation, pre-neoplasic transformation, in which EGFR may or may not have proven to play a key progression role. The term “cell proliferation disorder” also refers to all steps of cellular modifications, especially of the epidermis, including ones leading to epithelial thickening, preferably excluding tumor formation. The “cell proliferation disorder” can be associated with one or more diseases or disorders, including, but not limited to, CREST syndrome, corns and calluses, warts, hives, keratosis, atopic dermatitis, eczema, scleroderma, lipoderamtoscelerosis, an age spot or lentigo.

As used herein, “hyperplasia” refers to the increased cell production in a normal tissue or organ, specifically relating to an increase in number of cells. The term “hyperplasia” does not encompass cell proliferation disorders related to an increase in size of cells, nor tumor or cancerous changes of any cell, particularly epithelial cells.

One embodiment of the present invention relates to a method of preventing or inhibiting the progression of epidermal or epithelial hyperplasia in a subject, which comprises topically administering to the subject in need thereof a composition comprising an effective amount of an α adrenergic receptor agonist and a pharmaceutically acceptable carrier.

Another general aspect of the invention relates to a method of treating or reducing epidermal or epithelial hyperplasia in a subject. The method comprises topically administering to the subject in need thereof a composition comprising an effective amount of an α adrenergic receptor agonist and a pharmaceutically acceptable carrier.

As used herein, “epidermal or epithelial hyperplasia” refers to an abnormal increase in the number of skin cells in normal arrangement in organ or tissue, resulting in an increase in the organ or tissue volume. It can also be described as hypergenesis (i.e. an increase in the amount) of skin cells. Epidermal or epithelial hyperplasia can be triggered by anything from increased demand (i.e., to compensate for skin loss) to compensation for damage (i.e., an injury in the basal cell layer of skin or epithelium). Epidermal or epithelial hyperplasia is not associated with hypertrophy (i.e. an increase in size) of skin cells.

Another general aspect of the invention relates to a method of treating or reducing keratinocyte proliferation. The method comprises topically administering to the subject in need thereof a composition comprising an effective amount of an α adrenergic receptor agonist and a pharmaceutically acceptable carrier.

Another general aspect of the invention relates to a method of preventing or inhibiting keratinocyte proliferation. The method comprises topically administering to the subject in need thereof a composition comprising an effective amount of an α adrenergic receptor agonist and a pharmaceutically acceptable carrier.

As used herein, the term “keratinocyte proliferation” refers to an increase in the number of keratinocytes in the epidermis and basal layers of the skin.

As used herein, “inhibit” or “inhibiting” refers to a reduction of the progression of cell proliferation disorder.

As used herein, an “effective amount of an α adrenergic receptor agonist” with respect to reducing or inhibiting the progression of a cell proliferation disorder in a subject, means the amount of the α adrenergic receptor agonist that is sufficient to prevent or delay the progression of the cell proliferation disorder in a subject.

Another general aspect of the present invention relates to a method of regulating an EGFR response in a subject to thereby result in treating or preventing of a disease or condition associated with EGFR in a subject, comprising administering to the subject in need thereof a composition comprising an effective amount of an α adrenergic receptor agonist and a pharmaceutically acceptable carrier.

As used herein, a “disease or condition associated with EGFR” can be any disease or condition that can be treated by regulating the activity of EGFR. Preferably, a “disease or condition associated with EGFR” is not associated with a skin tumor, which includes a skin cancer, a benign skin tumor and pre-malignant skin tumor, nor is the “disease or condition associated with EGFR” associated with rosacea, erythema, telangiectasias psoriasis, purpura, sagging skin or wrinkle. Examples of “disease or condition associated with EGFR” include non-skin tumors, such as tumors of the oral cavity, head and neck tissues, esophagus, including local and metastatic tumors located in these tissues; and other cell proliferative disorders, such as cell proliferation disorder. The term “disease or condition associated with EGFR” also encompasses hyperplasia, increased proliferation, and pre-neoplasic lesion.

According to the present invention, in a method of regulating an EGFR response in a subject, the α adrenergic receptor agonist can be administered to the subject through any route of administration, including, but not limited to topical, epicutaneous, transdermal, subcutaneous, or intramuscular deliveries.

In a preferred embodiment, the α adrenergic receptor agonist is delivered to a skin area subject to UV-irradiation or chemotherapy-induced damages by topical application on the skin.

One embodiment of the present invention relates to a method of regulating EGFR driven epithelial pathologies related to increased proliferation in a subject, which comprises topically administering to the subject a composition comprising an effective amount of an α2 adrenergic receptor agonist and a pharmaceutically acceptable carrier.

As used herein, “regulate” or “regulating” refers to achieving a controlled response after application of the composition.

As used herein, “EGFR” refers to Epidermal Growth Factor Receptor, the cell-surface receptor for members of the epidermal growth factor family. EGFR is a member of the ErbB family of receptors of which there are four: EGFR, also referred to as ErbB1 or HER1; ErbB2 or HER2/c-neu; ErbB3 or HER3; and ErbB4 or HER4.

As used herein, an “effective amount of an α adrenergic receptor agonist” with respect to regulating an EGFR response in a subject, means the amount of the α adrenergic receptor agonist that is sufficient to regulate EGFR response such that a disease or condition in a subject is prevented or treated.

Another general aspect of the present invention relates to a method of treating hand and foot syndrome (HFS) in a subject in need thereof. The method comprises topically administering to a skin area of the subject a topical composition comprising an effective amount of at least one alpha adrenergic receptor agonist and a pharmaceutically acceptable carrier, wherein the skin area has, or is prone to have, hand and foot syndrome.

As used herein, “hand and foot syndrome” refers to reddening, swelling, numbness or desquamation on palms of the hands, soles of the feet, knees, elbows, elsewhere, or a combination thereof in a subject that occurs in response to a biological stimulus, or as a symptom of a disease. For example, “hand and foot syndrome” can occur from the use of a chemotherapy drug, or as a symptom of sickle-cell disease. In some cases hand and foot syndrome can also be referred to as acral erythema peculiar acral erythema, chemotherapy-induced acral crythema, palmar-plantar erythrodysesthesia, palmoplantar erythrodysesthesia, toxic erythema of the palms and soles, and Burgdorf's reaction.

In a preferred embodiment, the α adrenergic receptor agonist is delivered to a skin area susceptible to chemotherapy-induced hand and foot syndrome by topical application to the skin area.

As used herein, “chemotherapy-induced hand and foot syndrome” refers to an onset of the hand and foot syndrome in a subject as a result of use of a chemotherapy drug or other targeted cancer therapy. Chemotherapy and targeted cancer therapy drugs can include, but are not limited to, cytarabine, doxorubicin, fluorouracil, capecitabine, sorafenib, sunitinib.

In a method according to an embodiment of the present invention, the α adrenergic receptor agonist can be applied to the susceptible skin area prior to, after, or both prior to and after, the chemotherapy treatment.

As used herein, an “effective amount of an α adrenergic receptor agonist” with respect to reducing, inhibiting, or treating hand and foot syndrome in a subject, means the amount of the α adrenergic receptor agonist that is sufficient to prevent or reduce one or more symptoms associated with hand and foot syndrome, such that the hand and foot syndrome in a subject is prevented or treated.

Underlying causes of chemotherapy-induced hand and foot syndrome are unknown, however theories include temperature differences, vascular anatomies, and cell-type differences in the extremities, and are based on the fact that most or all of the symptoms are observed in the hands and feet of the subject. Merely as an observation, and without wishing to be bound by any theories, it is observed that chemotherapy-induced hand and foot syndrome is associated with one or more of the following indicators in the affected skin areas:

(a) erythema and oedema;

(b) accumulation of cytotoxic drugs in the skin due to a high level of vessels;

(c) drug metabolite excretion by sweat glands;

(d) greater activity of rate-limiting enzyme in the metabolism of pyrimidine analogues in keratinocytes; and

(e) abnormalities affecting the basal keratinocytes.

Another general aspect of the present invention relates to a method of treating cutaneous T-cell lymphomas (CTCL) in a subject in need thereof. The method comprises topically administering to a skin area of the subject a topical composition comprising an effective amount of at least one alpha adrenergic receptor agonist and a pharmaceutically acceptable carrier, wherein the skin area has, or is prone to have, CTCL.

As used herein, the term “cutaneous T-cell lymphomas” or “CTCL” refers to a skin condition in which there is an abnormal neoplastic proliferation of T lymphocytes. CTCLs are characterized by accumulation of malignant T cells in the skin. Early disease resembles benign skin disorders but during disease progression cutaneous tumors develop, and eventually the malignant T cells can spread to lymph nodes and internal organs. CTCL typically presents with red, scaly patches or thickened plaques of skin that often mimic eczema or chronic dermatitis. Progression from limited skin involvement is variable and can be accompanied by tumor formation, ulceration, and exfoliation, complicated by itching and infections.

In a preferred embodiment, the α adrenergic receptor agonist is delivered to a skin area susceptible to CTCL by topical application to the skin area. The α adrenergic receptor agonist can be applied to the skin area alone, or in combination with another treatment for CTCL, such as a treatment with Denileukin diftitox (Ontak), an engineered protein combining interleukin-2 and Diphtheria toxin; Bexarotene (Targretin), a retinoid; Vorinostat (Zolinza), a hydroxymate histone deacetylase (HDAC) inhibitor; or Romidepsin (Istodax), a cyclic peptide histone deacetylase (HDAC) inhibitor, etc.

The therapeutic effectiveness of an α adrenergic receptor agonist against CTCLs can be evaluated using various methods known in the art. For example, xenograft animal model of tumor stage CTCL can be used. Malignant T cells derived from CTCLs, such as MyLa2059, or HUT78 cells, are subcutaneously transplanted to an immune deficient mouse, such as NOD/SCID-B2m(-/-) (NOD.Cg-Prkdc(scid) B2m(tm1 Unc) /J), or CB-17 SCID beige mouse. Tumor growth at the xenograft site is monitored with or without the treatment. See, e.g., Krejsgaard, et al., 2010, 19(12):1096-102; Doebbeling Journal of Experimental & Clinical Cancer Research 2010, 29:11, both are incorporated herein by reference.

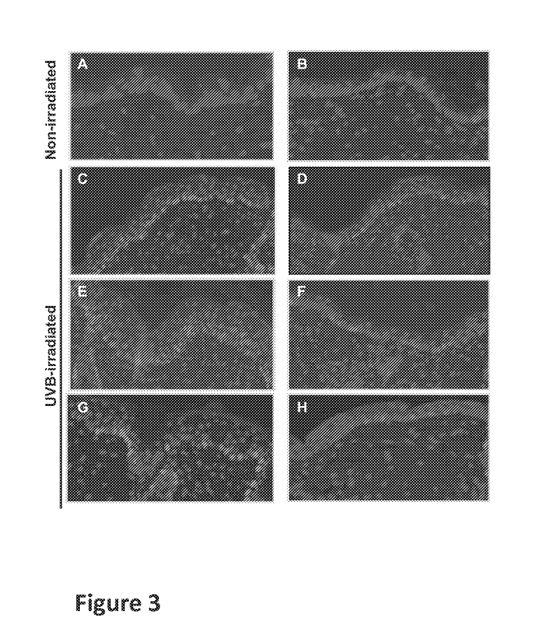

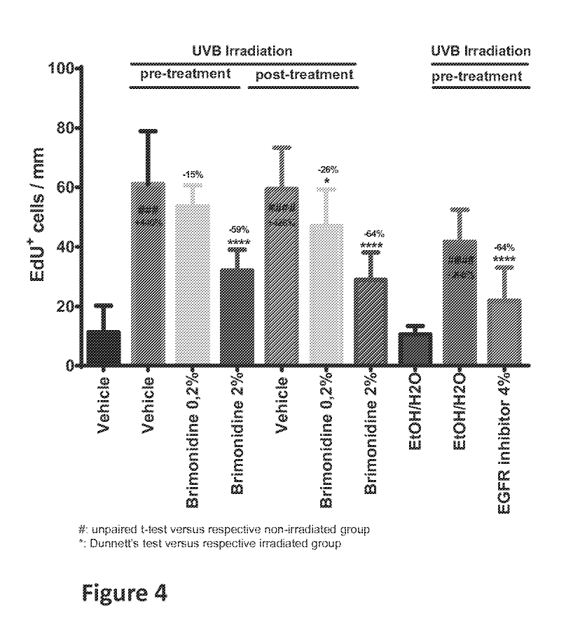

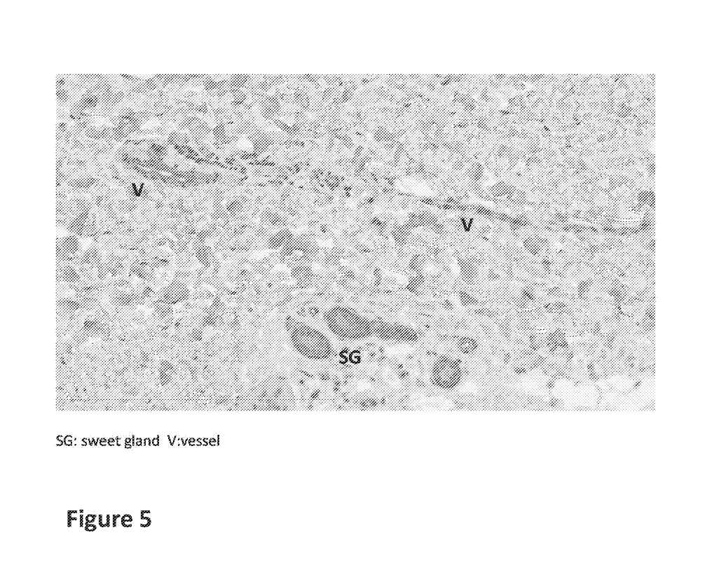

Any or all of the aforementioned indicators can be treated with an α adrenergic receptor agonist, such as brimonidine. Briefly, erythema and oedema have been shown to be mitigated by topical application of an α adrenergic receptor agonist, as described in U.S. Pat. No. 8,410,102, incorporated herein by reference. Brimonidine is a vasoconstrictor with anti-inflammatory properties (Piwnica et al., Vasoconstriction and anti-inflammatory properties of the selective α-adrenergic receptor agonist brimonidine. J Dermatol Sci. 2014 July;75(1):49-54), that can relieve the high level of vessels and the greater activity of rate-limiting enzymes in the metabolism of pyrimidine analogues. Brimonidine and other α adrenergic receptor agonists can decrease the sweat gland activity and the toxicity mediated by the metabolite excretion from the sweat glands, as sweat glands on human skin contain alpha2α adrenergic receptors (see e.g., FIG. 5). Finally, brimonidine can mitigate abnormalities affecting basal keratinocytes, as further explained in Example 6 and the results shown in FIGS. 3 and 4.

One skilled in the art will recognize that the effective amount of the α adrenergic receptor agonist to be used in the instant invention can vary with factors, such as the particular subject to be treated, e.g., age, diet, health, etc., degree of UV irradiation exposed to, dosage or particular chemotherapy drug exposed to, severity and complications of the cell proliferation disorder sought to be treated or inhibited, the α adrenergic receptor agonist used, the formulation used, etc. In view of the present disclosure, standard procedures can be performed to evaluate the effect of the administration of a composition to a subject, thus allowing a skilled artisan to determine the effective amount of the α adrenergic receptor agonist to be administered to the subject. Such effect can be, for example, a clinically observable beneficial effect of the a adrenergic receptor agonist in reducing or inhibiting the progression of cell proliferation disorder in a subject, or an in vivo or in vitro measurement on the EGFR activity, etc.

The clinically observable beneficial effect can be a situation that, when a composition of the present invention is administered to a subject after signs and/or symptoms, such as those related to cell proliferation disorder, are observable, the signs and/or symptoms are prevented from further development or aggravation, or develop to a lesser degree than without administration of the specified composition according to embodiments of the present invention. The clinically observable beneficial effect can also be that, when a composition of the present invention is administered to a subject before signs and/or symptoms, such as that related to cell proliferation disorder, are observable, the signs and/or symptoms are prevented from occurring or subsequently occur to a lesser degree than without administration of the composition of the present invention.

Methods of the present invention can be used in conjunction with one or more other treatments or medications for preventing or inhibiting the progression of skin or epithelium thickening, or treating existing signs and/or symptoms of skin or epithelium thickening. Examples of such other treatments or medications include, but are not limited to, retinoid and its derivatives, sun-screens or sun-blocks, anti-inflammatory agents, vitamins, such as vitamin D, nitroglycerin, etc.

Methods of the present invention can also be used in conjunction with one or more other treatments or medications for regulating an EGFR response in a subject, such as another anti-proliferative agent.

The other medicament or treatment can be administered to the subject simultaneously with, or in a sequence and within a time interval of, the administration of the α adrenergic receptor agonist, such that the active ingredients or agents can act together to treat or prevent cell proliferation disorder and signs and/or symptoms associated therewith. For example, the other medicament or treatment and the α adrenergic receptor agonist can be administered in the same or separate formulations at the same or different times.

Any suitable route of administration can be employed to deliver the additional treatment or medication including, but not limited to, oral, intraoral, rectal, parenteral, topical, epicutaneous, transdermal, subcutaneous, intramuscular, intranasal, sublingual, buccal, intradural, intraocular, intrarespiratory, or nasal inhalation.

A composition according to embodiments of the present invention comprises an effective amount or a therapeutically effective amount of an α adrenergic receptor agonist and a pharmaceutically acceptable carrier.

The carriers useful for topical delivery of the specified compounds according to embodiments of the invention can be any carrier known in the art for topically administering pharmaceuticals, including, but not limited to, pharmaceutically acceptable solvents, such as a polyalcohol or water; emulsions (either oil-in-water or water-in-oil emulsions), such as creams or lotions; micro emulsions; gels; ointments; liposomes; powders; and aqueous solutions or suspensions. The pharmaceutically acceptable carrier includes necessary and inert pharmaceutical excipients, including, but not limited to, binders, suspending agents, lubricants, flavorants, preservatives, dyes, and coatings.

The topical composition according to embodiments of the present invention are prepared by mixing a pharmaceutically acceptable carrier with a therapeutically effective amount of an α2 adrenergic receptor agonist according to known methods in the art, for example, methods provided by standard reference texts such as, REMINGTON: THE SCIENCE AND PRACTICE OF PHARMACY 1577-1591, 1672-1673, 866-885 (Alfonso R. Gennaro ed. 19th ed. 1995); TRANSDERMAL AND TOPICAL DRUG DELIVERY SYSTEMS (Ghosh, T. K., et al. ed. 1997), both of which are hereby incorporated herein by reference.

In one embodiment, the topical composition of the invention is in the form of an emulsion. Emulsions, such as creams and lotions are suitable topical formulations for use in the invention. An emulsion is a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase, the emulsion is termed an oil-in-water emulsion. Emulsions, such as creams and lotions that can be used as topical carriers and their preparation are disclosed in REMINGTON: THE SCIENCE AND PRACTICE OF PHARMACY 282-291 (Alfonso R. Gennaro ed. 19th ed. 1995), hereby incorporated herein by reference.

In another embodiment, the topical composition of the invention is in the form of a gel, for example, a two-phase gel or a single-phase gel. Gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are disclosed in REMINGTON: THE SCIENCE AND PRACTICE OF PHARMACY 1517-1518 (Alfonso R. Gennaro ed. 19th ed. 1995), hereby incorporated herein by reference. Other suitable gels for use with the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002), each of which patents is hereby incorporated herein by reference.

In an embodiment, the topical composition further comprises an aqueous gel comprising water and a water-gelling amount of a pharmaceutically acceptable gelling agent selected from the group consisting of carbomers, glycerine polyacrylate, and mixtures thereof, and the topical composition has a physiologically acceptable pH.

As used herein, “carbomer” is the USP designation for various polymeric acids that are dispersible but insoluble in water. When the acid dispersion is neutralized with a base a clear, stable gel is formed. Carbomer 934P is physiologically inert and is not a primary irritant or sensitizer. Other carbomers include 910, 940, 941, and 1342. Polymer thickeners (gelling agents) that may be used in compositions according to embodiments of the present invention include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). Preferably the gelling agent comprises between about 0.2% to about 4% by weight of the composition. More particularly, the preferred compositional weight percent range for “CARBOPOL®” is between about 0.5% to about 2%, while the preferred weight percent range for “NATROLSOL®” and “KLUCEL®” is between about 0.5% to about 4%. The preferred compositional weight percent range for both “HYPAN®” and “STABILEZE®” is between 0.5% to about 4%.

“CARBOPOL®” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

In another preferred embodiment, the topical composition of the invention is in the form of an ointment. Ointments are oleaginous semisolids that contain little if any water. Preferably, the ointment is hydrocarbon based, such as a wax, petrolatum, or gelled mineral oil. Suitable ointments for use in the invention are well known in the art and are disclosed in REMINGTON: THE SCIENCE AND PRACTICE OF PHARMACY 1585-1591 (Alfonso R. Gennaro ed. 19th ed. 1995), hereby incorporated herein by reference.

In an embodiment of the present invention, the topical composition of the invention comprises at least one of a cream and an ointment, each comprising an agent selected from the group consisting of stearic acid, stearyl alcohol, cetyl alcohol, glycerin, water, and mixtures thereof, and the topical composition has a physiologically acceptable pH.

In another embodiment, the topical composition of the invention is in the form of an aqueous solution or suspension, preferably, an aqueous solution. Suitable aqueous topical formulations for use in the invention include those disclosed in REMINGTON: THE SCIENCE AND PRACTICE OF PHARMACY 1563-1576 (Alfonso R. Gennaro ed. 19th ed. 1995), hereby incorporated herein by reference. Other suitable aqueous topical carrier systems include those disclosed in U.S. Pat. No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); and U.S. Pat. No. 6,465,464 (issued Oct. 15, 2002), all of which patents are hereby incorporated herein by reference.

The pH of the topical formulations of the invention are preferably within a physiologically acceptable pH, e.g., within the range of about 5 to about 8, more preferably, of about 5.5 to about 6.5. To stabilize the pH, preferably, an effective amount of a buffer is included. In one embodiment, the buffering agent is present in the aqueous topical formulation in an amount of from about 0.05 to about 1 weight percent of the formulation. Acids or bases can be used to adjust the pH as needed.

Tonicity-adjusting agents can be included in the aqueous topical formulations of the invention. Examples of suitable tonicity-adjusting agents include, but are not limited to, sodium chloride, potassium chloride, mannitol, dextrose, glycerin, and propylene glycol. The amount of the tonicity agent can vary widely depending on the formulation's desired properties. In one embodiment, the tonicity-adjusting agent is present in the aqueous topical formulation in an amount of from about 0.5 to about 0.9 weight percent of the formulation.

Preferably, the aqueous topical formulations of the invention have a viscosity in the range of from about 15 cps to about 25 cps. The viscosity of aqueous solutions of the invention can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose.

In a preferred embodiment, the aqueous topical formulation of the invention is isotonic saline comprising a preservative, such as benzalkonium chloride or chlorine dioxide, a viscosity-adjusting agent, such as polyvinyl alcohol, and a buffer system such as sodium citrate and citric acid.

The topical composition according to embodiments of the invention can comprise pharmaceutically acceptable excipients such as those listed in REMINGTON: THE SCIENCE AND PRACTICE OF PHARMACY 866-885(Alfonso R. Gennaro ed. 19th ed. 1995); and TRANSDERMAL AND TOPICAL DRUG DELIVERY SYSTEMS (Ghosh, T. K. et al. ed. 1997), hereby incorporated herein by reference, including, but not limited to, protectives, adsorbents, demulcents, emollients, preservatives, antioxidants, moisturizers, buffering agents, solubilizing agents, skin-penetration agents, and surfactants.

In an embodiment, the topical composition of the invention further comprises one or more agents selected from the group consisting of a preservative, a local anesthetic and a skin humectant.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; antibacterial esters, for example, esters of parahydroxybenzoic acid; and other anti-microbial agents such as chlorhexidine, chlorocresol, benzoic acid and polymyxin.

The topical composition according to embodiments of the invention can include pharmaceuticals or their pharmaceutically acceptable salts, such as an α2 adrenergic receptor agonist, and optionally one or more other pharmaceutically active ingredients, including, but not limited to, corticosteroids and other anti-inflammatory agents, such as betamethasone, diflorasone, amcinonide, fluocinolone, mometasone, hydrocortisone, prednisone, and triamcinolone; local anesthetics and analgesics, such as camphor, menthol, lidocaine, and dibucaine, and pramoxine; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and anti-infectives, such as mupirocin, erythromycin, clindamycin, gentamicin, polymyxin, bacitracin, and silver sulfadiazine; and antiseptics, such as iodine, povidine-iodine, benzalkonium chloride, benzoic acid, chlorhexidine, nitrofurazine, benzoyl peroxide, hydrogen peroxide, hexachlorophene, phenol, resorcinol, and cetylpyridinium chloride.

In a preferred embodiment, a topical composition according to embodiments of the invention further comprises titanium dioxide (TiO₂), preferably at an amount that is sufficient to mask the color of brimonidine or another colored ingredient in the formulation, but would not cause irritation to the skin. TiO₂ may cause mild irritation and reddening to the eyes, thus eye contact with the TiO₂—containing topically administrable composition should be avoided.

Dosages and dosing frequency will be determined by a trained medical professional depending on the activity of the compounds used, the characteristics of the particular topical formulation, and the identity and severity of the dermatologic disorder treated or prevented.

In one embodiment of the invention, the topical composition is applied in one or more applications, for example the topical composition can be applied in one, two, three, four, or more applications. According to the invention, the topical composition can be applied in one or more applications before, one or more applications after, or one or more applications before and after the UV-irradiation or chemotherapy. The one or more applications before and after include any combination of applications, for example one application before UV-irradiation or chemotherapy, followed by multiple applications after UV-irradiation or chemotherapy, or vice versa. The one or more applications can further be spaced by any amount of time. For example, an application before UV-irradiation or chemotherapy can be applied more than one hour before, about one hour before, less than one hour before, or immediately before the UV-irradiation or chemotherapy. Likewise, an application after UV-irradiation or chemotherapy can be applied immediately after, less than one hour after, about one hour after, or more than one hour after the UV-irradiation or chemotherapy. Subsequent applications can be applied any time after the previous application. For example about one hour after the application, 22 hours after the application, 23 hours after the application, 24 hours after the application, etc.

As used herein, the terms “immediately before” and “immediately after” with respect to an application of the topical composition include any time less than one hour before or one hour after chemotherapy. Preferably, “immediately before” and “immediately after” refer to any time 20 minutes or less before UV-irradiation or chemotherapy, or any time 20 minutes or less after UV-irradiation or chemotherapy. For example, any time immediately after UV-irradiation or chemotherapy can be 1, 2, 3, 4, 5, 6, 7, 8, 9, 10, 11, 12, 13, 14, 15, 16, 17, 18, 19, or 20 minutes after UV-irradiation or chemotherapy.

In an embodiment of the present invention, the topical composition comprises 0.01% to 5% by weight of an α adrenergic receptor agonist, such as an α2 adrenergic receptor agonist. The composition can comprise from about 0.1%, 0.2%, 0.3%, 0.4%, 0.5%, 0.6%, 0.7%, 0.8%, 0.9% or 1.0% by weight of the alpha adrenergic receptor agonist. For example, the composition can comprise, 0.01%, 0.05%, 0.1%, 0.2%, 0.3%, 0.4%, 0.5%, 0.6%, 0.7%, 0.8%, 0.9%, 1%, 2%, 3%, 4% or 5%, by weight, of the α2 adrenergic receptor agonist. In one embodiment, the topical composition comprises 0.1% to 0.6% by weight of the α2 adrenergic receptor agonist.

To prevent or inhibit cell proliferation disorder in view of the present disclosure, for example, the topical compositions of the invention are topically applied directly to the area exposed to sunlight or the otherwise affected area in any conventional manner well known in the art, e.g., by dropper or applicator stick, as a mist via an aerosol applicator, via an intradermal or transdermal patch, or by simply spreading a formulation of the invention onto the affected area with fingers. Generally the amount of a topical formulation of the invention applied to the affected skin area ranges from about 0.1 g/cm² of skin surface area to about 5 g/cm², preferably, 0.2 g/cm² to about 0.5 g/cm² of skin surface area. Typically, one to four applications per day are recommended during the term of treatment.

The topical formulations of the invention can be filled and packaged into a plastic squeeze bottle or tube. Suitable container-closure systems for packaging a topical formulation of the invention are commercially available for example, from Wheaton Plastic Products, 1101 Wheaton Avenue, Millville, N.J. 08332.

Preferably, instructions are packaged with the formulations of the invention, for example, a pamphlet or package label. The labeling instructions explain how to administer topical formulations of the invention, in an amount and for a period of time sufficient to prevent or inhibit cell proliferation disorder and signs and/or symptoms associated therewith. Preferably, the label includes the pharmacology, drug resistance, pharmacokinetics, absorption, bioavailability, and contraindications.

This invention will be better understood by reference to the non-limiting examples that follow, but those skilled in the art will readily appreciate that the examples are only illustrative of the invention as described more fully in the claims which follow thereafter.

### EXAMPLE 1

### Aqueous Topical Formulations

This example illustrates aqueous topical formulations that can be used in the present invention.

A first aqueous solution topical formulation comprises: brimonidine tartrate (0.01% to 5% w/w); Puriteg (0.005% w/w) (stabilized chlorine dioxide) as a preservative; and the inactive ingredients: boric acid; calcium chloride; magnesium chloride; potassium chloride; purified water; sodium borate; sodium carboxymethylcellulose; sodium chloride; with hydrochloric acid and/or sodium hydroxide to adjust the pH to 5.6 to 6.6. The osmolality is in the range of 250-350 mOsmol/kg.

A second aqueous solution topical formulation comprises brimonidine tartrate (0.2% to 2% w/w); benzalkonium chloride (0.005% w/w.) as a preservative; and the inactive ingredients: boric acid; calcium chloride; magnesium chloride; potassium chloride; purified water; sodium borate; sodium carboxymethylcellulose; sodium chloride; with hydrochloric acid and/or sodium hydroxide to adjust the pH to 5.6 to 6.6. The osmolality is in the range of 250-350 mOsmol/kg.

### EXAMPLE 2

### Cream or Ointment Topical Formulations

This example illustrates cream or ointment topical formulations that can be used in the present invention.

A first cream topical formulation (hydrophilic ointment) is described in Table 2 below.

TABLE 2

| Ingredient | Weight Percent |
|---|---|
| Brimonidine tartrate | 0.01% to 5% |
| Stearic acid | 7% |
| Stearyl alcohol | 5% Cetyl alcohol |
| 2% Glycerin | 10% |
| Sodium lauryl sulfate | 1% |
| Propylparaben | 0.05% |
| Methylparaben | 0.25% |
| Disodium edetate | 0.055% |
| Distilled water | QS |
| TOTAL | 100% |

To make the formulation, the stearyl alcohol and the white petrolatum are melted on a steam bath, and warmed to about 75 degrees C. The other ingredients, previously dissolved in the water and warmed to 75 degrees C., are then added, and the mixture is stirred until it congeals. The mixture is then allowed to cool with stirring, and brimonidine tartrate is then added as a concentrated solution.

An ointment topical formulation (hydrophilic ointment) is described in Table 3 below.

TABLE 3

| Ingredients | Weight | |
|---|---|---|
| Brimonidine tartrate | 20 | g |
| Cholesterol | 30 | g |
| Stearyl Alcohol | 30 | g |
| White Wax | 80 | g |
| White Petrolatum | 820-800 | g |

To make the formulation, the stearyl alcohol and white wax are mixed together on a steam bath. The cholesterol is then added and stirred until it completely dissolved. The white petrolatum is then added and mixed. The mixture is removed from the bath, and stirred until it congeals. With continuous stirring, brimonidine tartrate is added as a concentrated slurry.

### EXAMPLE 3

### Gel Topical Formulations

This example illustrates gel topical formulations that can be used in the present invention.

A first gel formulation is described in Table 4 below.

TABLE 4

| Ingredients | Weight % |
|---|---|
| Brimonidine tartrate | 0.01-5% |
| Methylparaben NF | 0.15% |
| Propylparaben NF | 0.03% |
| Hydroxyethylcellulose NF | 1.25% |
| Disodium Edetate USP | 0.05% |
| Purified Water, USP | QS |
| TOTAL | 100% |

A second gel formulation is described in Table 5 below.

TABLE 5

| Ingredients | Weight % |
|---|---|
| Brimonidine tartrate | 0.5% |
| Methylparaben | 0.20% |
| Propylparaben | 0.05% |
| Carbomer 934P NF | 1.0% |
| Sodium Hydroxide | QS pH 7 |
| Purified Water, USP | QS |
| TOTAL | 100% |

The ingredients are mixed together and aqueous sodium hydroxide is slowly added to the mixture until a pH of about 7 is reached and the gel is formed.

A third gel formulation is described in Table 6 below.

TABLE 6

| Ingredient | Weight Percent |
|---|---|
| Brimonidine tartrate | 0.18% |
| Carbomer 934P | 1.25% |
| Methylparaben | 0.3% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

A fourth gel formulation is described in Table 7 below.

TABLE 7

| Ingredients | Weight % |
|---|---|
| Brimonidine tartrate | 0.01-5% |
| Methylparaben | 0.2% |
| Propylparaben | 0.05% |
| “CARBOPOL ®” | 1.0% |
| Triethanolamine | QS pH 7 |
| Water | QS |
| TOTAL | 100% |

The ingredients are mixed together and stirred. Triethanolamine is added until a pH of about 7 is attained.

### EXAMPLE 4

### Foam Topical Formulations

This example illustrates foam topical formulations that can be used in the present invention.

A first foam formulation is described in Table 8 below.

TABLE 8

| Ingredients | Amount (Weight %) |
|---|---|
| Brimonidine tartrate | 0.01-5 |
| Stearic Acid | 4.2 |
| Laureth-23 | 1.4 |
| Sodium Lauryl Sulfate | 0.5 |
| Triethanolamine | 2.2 |
| Butylated hydroxytoluene (BHT) | 0.01 |
| Fragrance | 0.5 |
| Aeron A-31 Propellant | 3 |
| Water | QS |
| TOTAL | 100 |

The water is heated to 80-85° C., after which stearic acid is added. Once the stearic acid is melted, the laureth-2.3 is added, melted, and mixed well. Next, triethanolamine is added and the resulting composition is mixed well for about 30 minutes to form a soap. The resulting soap is then cooled to about 65° C., after which sodium lauryl sulfate is added. The composition is then mixed well. Next, the BHT and the Brimonidine tartrate are added, followed by mixing. The resulting composition is then cooled to room temperature and the fragrance added. The product is packaged with the Acron A-31 propellant in an aerosol can using conventional techniques and mechanically shaken for 5 minutes. The product dispenses as a cone-shaped spray that deposits onto the skin as a layer of rich lather that quickly covers a wide area of skin, and begins to relieve symptoms within about 2 minutes after application.

A second foam formulation is described in Table 9 below.

TABLE 9

| Ingredient | Amount (Weight %) |
|---|---|
| Brimonidine tartrate | 0.2-2 |
| Water | QS |
| Palmitic Acid | 2.12 |
| Laureth-23 | 0.93 |
| Triethanolamine (99%) | 1.13 |
| Cetyl Dimethicone Copolyol | 0.19 |
| Mineral Oil | 0.31 |
| Stearyl Alcohol | 0.31 |
| Lauramide DEA | 0.15 |
| PEG-150 Distearate | 0.05 |
| Imidazolidinyl Urea | 0.0016 |
| Methylparaben | 0.0005 |
| Propylparaben | 0.00003 |
| Freeze Dried Aloe Powder | 0.0015 |
| Fragrance | 0.50 |
| Aeron A-31 Propellant | 3.00 |
| TOTAL | 100 |

The aqueous phase is prepared as follows. The water is heated to 80° C., after which palmitic acid is added. Once the palmitic acid is melted, the laureth-23 is added, melted, and mixed well. Next, triethanolamine is added and the resulting composition is mixed well for about 15 minutes to form a soap.

Stearyl alcohol, mineral oil, lauramide DEA, cetyl dimethicone copolyol, PEG-1 50 distearate, and BHT are mixed and heated at 55° C. to form the oil phase. The oil phase is combined with the aqueous phase at 80° C. and mixed well for about 15 minutes. The resulting mixture is then cooled to room temperature and the imidazolidinyl urea, methylparaben, and propylparaben are added, and then mixed well. The brimonidine tartrate is then added, and mixed well. Next, the fragrance is added, followed by gentle mixing. The aloe is then dissolved in make-up water and added with slow mixing to form the product formulation which is then packaged in an aerosol can as described for the first foam formulation.

The product dispenses as a cone-shaped spray that deposits onto the skin as a layer of rich lather that quickly covers a wide area of skin, and begins to relieve symptoms within about 2 minutes after application.

A third non-soapy foam formulation is described in Table 10 below.

TABLE 10

| Ingredient | Amount (Weight %) |
|---|---|
| Brimonidine tartrate | 0.4-0.6 |
| Ethanol | 6 |
| Ethyl Ester of PVM/MA | 4 |
| Copolymer Dimethicone Copolyol | 0.1 |
| Water | QS |
| PVP/VA Copolymer | 1 |
| Sodium Lauryl Sulfate | 1 |
| Oleth-20 | 0.5 |
| Cocamide MEA | 0.05 |
| Methyl Paraben | 0.1 |
| Aminomethyl Propanol | 0.53 |
| Stearalkonium Chloride | 0.05 |
| Steareth-16 | 0.1 |
| Panthenol | 0.5 |
| Fragrance | 0.5 |
| Aeron A-465 | 5 |
| TOTAL | 100 |

The alcohol phase is prepared by dissolving ethyl ester of PVM/MA copolymer in ethanol, after which dimethicone is added and mixed well. The aqueous phase is prepared by heating the water to 65° C., after which the PVP/VA copolymer is added and mixed well. The oil phase is prepared by mixing the oleth-20, cocamide MEA, and steareth-16 at 60° C. to form a blend. The oil phase is then added to the aqueous phase at 65° C. and mixed well. Next, the methylparaben is added to the mixture, followed by mixing, after which the aminomethyl propanol, stearalkonium chloride, and panthenol are added and mixed until uniform. The resulting composition is cooled to room temperature, after which the alcohol phase is added and mixed well. The fragrance is then added and mixed gently to form the product. The product is then packaged in an aerosol can.

The product dispenses as a cone-shaped spray that deposits onto the skin as a layer of rich lather that quickly covers a wide area of skin, and begins to relieve symptoms within about 2 minutes after application.

### EXAMPLE 5

### Photo Study with Brimonidine

Albino hairless SKH1-hr mice (36/sex/group) were treated for 40 weeks with UVR and brimonidine gel or vehicle according to the design in table 11. Mice were further observed for 12 weeks without treatment. Topical treatments were performed approximately one hour before UVR on Monday, Wednesday and Friday of each week and approximately one hour after UVR Tuesday and Thursday of each week. See table 11.

All procedures involving animals were conducted in a fully accredited animal facility and in accordance with the preapproved protocols.

TABLE 11

| | | | | | | Treatment |
|---|---|---|---|---|---|---|
| | | | Frequency of | Solar- | Duration of | free |
| | Brimonidine | Administration | administration | simulated | Treatment | follow-up |
| Dosage | tartrate | (μL/mouse, on | (days per | UVR dose | or exposure | period |
| Group | Conc. (%) | 25 cm² BSA) | wk)* | (RBU/week) | (weeks) | (weeks) |
| 1 | Vehicle | 100 | 5 | 600 | 40 | 12 |
| 2 | 0.18 | 100 | 5 | 600 | 40 | 12 |
| 3 | 1 | 100 | 5 | 600 | 40 | 12 |
| 4 | 2 | 100 | 5 | 600 | 40 | 12 |
| 5 | N/A | N/A | N/A | 600 | 40 | 12 |
| 6 | N/A | N/A | N/A | 1200 | 40 | 12 |

N/A: NOT APPLICABLE

BSA: body surface area

RBU: Robertson-Berger Unit (a measure of effectiveness of UVR; 400 RBU approximates one minimal erythema dose in previously untanned human skin)

*Monday, Wednesday and Friday of each week: exposure to UVR approximately one hour after test item application. Tuesday and Thursday of each week: exposure to UVR approximately one hour before test item application.

As the results shown in Table 12, topical application of brimonidine at 0.18%, 1%, and 2% concentrations surprisingly resulted in a dose-dependent reduction in UV-induced skin thickening. The UVR exposure was 600 RBU/week for all test groups in the table.

TABLE 12
Group Comparisons of Skin Thickening Prevalence

| Group | Vehicle | 0.18% | 1% | 2% | UV control |
|---|---|---|---|---|---|
| UV treatment | Yes | Yes | Yes | Yes | Yes |
| Male tested | 36 | 36 | 36 | 36 | 36 |
| Skin | 589/35 | 514/34 | 392/27** | 367/24** | 797/35 |
| Thickening | | | | | |
| Female tested | 36 | 36 | 36 | 36 | 36 |
| Skin | 554/33 | 521/32 | 374/25** | 381/23** | 577/34 |
| Thickening | | | | | |

**p < 0.01 compared to the vehicle control group

Total number of observations/number of mice with observation: 171 (males) and 181 females.

Although treatment with 0.18% (w/w) brimonidine did not statistically reduce the UV-induced skin thickness, the incidence of skin thickening in this treatment group was still observably lower than that in the UV control group. Both groups treatment with 1% and 2% (w/w) brimonidine statistically reduced the UV-induced skin thickness compared to the UV control group. The observed reduction was clearly related to brimonidine as the vehicle control group has no effect compared to the UV control group alone, indicating that an alpha adrenergic receptor agonist is effective in reducing skin thickening, such as that induced by UV.

While not wishing to be bound by theory, the observed reduction in the UV-induced skin thickening appeared to be due, at least in part, to the regulation of the EGFR response, e.g., by inhibiting the EGFR related keratinocyte proliferation or EGER related suppression of apoptosis when skin is stimulated by UV radiation. Thus, an alpha adrenergic receptor agonist can be used to regulate an EGFR response for the treatment of a disease or disorder associated with EGFR.

### EXAMPLE 6

### UVB-induced Epidermal Hyperplasia

Materials and Methods

SKH1 mice were treated and exposed to UVB irradiation. The back skin of the mice was exposed to 120 mJ/cm² UVB using the Biospectra system equipped with UVB sunlamps with a maximum emission peak at 312 nm. Irradiations were performed under isoflurane gaseous anesthesia. Mice were treated with vehicle (PEG400/EtOH/PHY (30/20/50) p/p) or brimonidine tartrate at 0.2% or 2%, by weight, administrated by topical application (100 μl) on a 10 cm² area on the upper part of the back using a micropipette. The area is located on the upper part of the back to prevent the animals from liking themselves. Three applications were performed according to the following two treatment schedules:

(a) pre-treatment: one application 1 hour prior to UVB irradiation, followed by two more applications 23 hours apart;

(b) post-treatment: one application immediately following the UVB irradiation, followed by two more applications 23 hours apart.

A compound reference, i.e., an EGFR inhibitor at 4%, by weight, in EtOH/H₂O (76/24) was also administrated according to the pre-treatment schedule.

One hour after the third topical treatment and one hour before euthanasia, mice received an intraperitoneal (i.p.) injection of 5-ethynyl-2′-deoxyuridine (EdU) at 100 mg/kg. After mouse euthanasia using cervical dislocation, the back skin was removed and immediately fixed in formol. The formol-fixed skin samples were embedded in paraffin and then submitted to immunohistological studies for epidermal thickness and EdU detection.

For epidermal thickness, two sections of 7 μm per animal were stained in hematoxylin and eosin (H&E) stain. Skin histology and the epidermis thickness were analyzed using image analysis (mScope 3.9, Aurora mScope) on scanned slide pictures (NanoZoomer C9600-12, Hamamatsu Photonics K.K).

For EdU detection, two or three sections of 7 μm per animal were submitted to EdU staining. Briefly, paraffin sections were rehydrated and were incubated 30 minutes with Click-iT® reaction cocktail (Click-iT® EdU Imaging Kit with Alexa Fluor® 594 Azide from Life Technologies). Nuclei were also stained with 4′,6-diamidino-2-phenylindole (DAPI) diluted at 5 mg/mL in Vectashield Hard Set Mounting medium for Fluorescence (ref: H-1400 Vector Laboratories). The number of keratinocytes stained with Alexa Fluor® 594 was counted from digital images (NanoZoomer C9600-12, Hamamatsu Photonics K.K) and the epidermis length of each section determined using an in house dedicated tool.

Data Analysis

Data were analyzed using unpaired t test for validation of the UVB irradiation and one-way ANOVA with Dunnett's multiple comparison test for the analysis of the treatment effect (Prism 6; GraphPad Software Inc., San Diego, Calif., USA). A p value of less than 0.05 was regarded as significant.

Results

The effect of brimonidine tartrate after topical administration on the murine UVB-induced epidermal hyperplasia model was evaluated.

The histological analysis of H&E stained skin sections showed that UVB irradiation at 120 mJ/cm² on mouse skin produces epidermal acanthosis (thickening of the skin) highly visible 48 hours following the irradiation (epidermis thickness increased by +127% in comparison with vehicle-treated mice). This acanthosis was inhibited fully (−96%) by topical pre-treatment with a reference treatment of an EGFR inhibitor at 4% (w/w) and partially (−23%) by the application of brimonidine tartrate at 2% (w/w), each was applied one hour before UVB irradiation and then twice more at 23 hours apart. However no effect was observed with a lower dose of 0.2% (w/w) brimonidine tartrate.

In order to exclude a UVB-filter effect of brimonidine and support a pharmacological activity, the same experiments were conducted according to the post-treatment schedule. Similar results were obtained in this case with a slight decrease of the epidermal thickness with 0.2% (w/w) brimonidine tartrate, and a largest and significant decrease with 2% (w/w) brimonidine tartrate (−16% and −32% respectively).

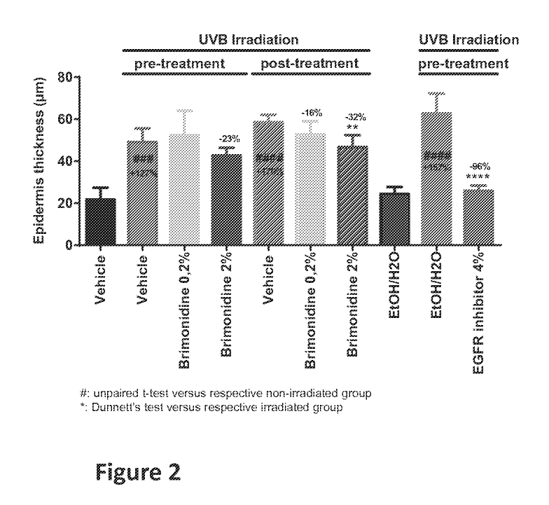

To better characterize the effect of brimonidine tartrate on epidermal hyperplasia, a proliferation marker was analyzed. EdU, a thymidine analogue, was incorporated into cellular DNA during DNA replication, and the incorporated EdU was detected through a “click” reaction with a fluorescent Alexa Fluor®594 azide (Zeng, Bain Res. 2010). It was confirmed that one UVB irradiation produces an increment of proliferating keratinocytes stained with Alexa Fluor®594 (more than 4 fold). As shown in FIG. 2, the reference compound EGFR inhibitor decreased by 64% the number of proliferating keratinocytes. Brimondine tartrate at 2% in pre- and post-treatment successfully reduced the number of EdU positive cells (−59% and −64% respectively). This effect was also observed with the lower dose, 0.2% brimonidine tartrate, in post-treatment (decrease of 25%).

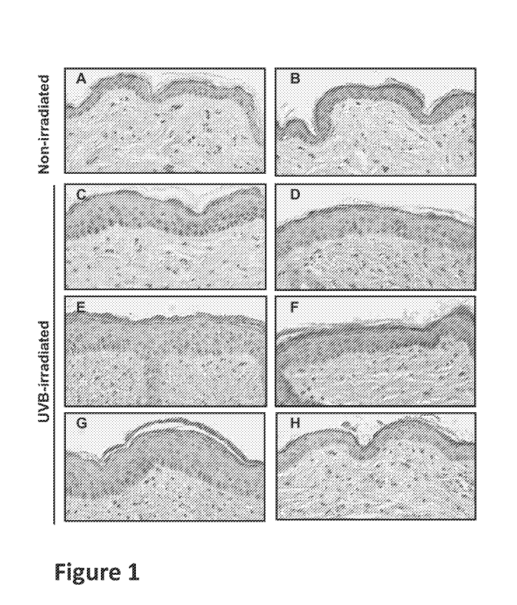

FIG. 1 shows the response of mouse skin to one UVB irradiation and the impact of 2% brimonidine tartrate treatment upon epidermis thickness. Acanthosis was inhibited by topical application of an EGFR inhibitor at 4% (w/w). H&E stained dorsal skin sections shown in FIG. 1 are as follows:

Section A: non irradiated skin treated with the vehicle;

Section B: non irradiated skin treated with EtOH/H₂O;

Section C: irradiated skin treated with vehicle according to the pre-treatment schedule;

Section D: irradiated skin treated with 2% (w/w) brimonidine tartrate according to the pre-treatment schedule;

Section E: irradiated skin treated with vehicle according to the post-treatment schedule;

Section F: irradiated skin treated with 2% (w/w) brimonidine tartrate according to the post-treatment schedule;

Section G: irradiated skin treated with EtOH/H₂O according to the pre-treatment schedule;

Section H: irradiated skin treated with an EGFR inhibitor at 4% (w/w) according to the pre-treatment schedule.

FIG. 2 is a quantification analysis of the effects of topical application of 0.2% and 2%, by weight, brimonidine tartrate according to the pre- and post-treatment schedules on epidermal thickness. The reduction in epidermal thickness caused by a post-treatment with 2% (w/w) brimonidine tartrate was statistically significant (**p<0.01). An EGFR inhibitor at 4% (w/w) was used a reference treatment (mean±SD).

FIG. 3 shows the response of mouse skin to one UVB irradiation and the impact of 2% (w/w) brimonidine tartrate treatment upon keratinocyte proliferation. Acanthosis was inhibited by topical application of an EGFR inhibitor at 4% (w/w). EdU staining (pink) and DAPI staining (blue) show keratinocyte and nuclei, respectively, in the sections shown in FIG. 3 as follows:

(A)non irradiated skin treated with the vehicle;

(B) non irradiated skin treated with EtOH/H₂O;

(C) irradiated skin treated with vehicle according to the pre-treatment schedule;

(D) irradiated skin treated with 2% (w/w) brimonidine tartrate according to the pre-treatment schedule;

(E) irradiated skin treated with vehicle according to the post-treatment schedule;

(F) irradiated skin treated with 2% (w/w) brimonidine tartrate according to the post-treatment schedule;

(G) irradiated skin treated with EtOH/H₂O according to the pre-treatment schedule;

(H) irradiated skin treated with an EGFR inhibitor at 4% (w/w) according to the pre-treatment schedule.

FIG. 4 is a quantification analysis of the effects of topical application of 0.2% and 2%, by weight, brimonidine tartrate according to the pre- and post-treatment schedules on epidermal proliferation. EdU incorporation was calculated as the number of EdU positive cells in the basal layer of the epidermis relative to the epidermis length. The reduction in epidermal proliferation caused by a treatment with brimonidine tartrate was statistically significant (*p<0.05 and ****p<0.0001) for both doses (0.2% and 2% brimonidine tartrate) administered according to the post-treatment schedule and for the 2% brimonidine tartrate dose administered according to the pre-treatment schedule. An EGFR inhibitor at 4% (w/w) was used a reference treatment (mean±SD).

### EXAMPLE 7

### Immunohistochemistry

In a human skin biopsy, Alpha2a adrenergic receptor was detected by immunohistochemistry on vessels and on sweat glands, as it is shown in FIG. 5.

Cut sections from formalin-fixed paraffin-embedded tissues were prepared. These sections were dewaxed, and Alpha2a adrenergic receptor was detected with a polyclonal antibody (Abcam, dilution 1:10). The antibody was detected with an Ultravision detection system, AEC substrate (Termofisher). Sections were counterstained with hematoxylin and mounted.

### EXAMPLE 8

### Treatment in a Hand and Foot Syndrome Animal Model

Protocol for Preparation of a Chemotherapy-induced HFS Animal Model

A female SD rat is used to prepare a chemotherapy-induced HFS animal model, according to the protocol described in Yokomichi (Yokomichi et al., Pathogenesis of Hand-Foot Syndrome induced by PEG-modified liposomal Doxorubicin, Hum Cell. March 2013; 26(1): 8-18), which is herein incorporated by reference in its entirety. Briefly, a PEG-modified liposomal doxorubicin formulation (PEGL-DOX), a chemotherapy drug commonly used against recurrent ovarian cancer, is administered at 5 or 10 mg/kg to seven week old female SD rats via tail vain injection once every 3 days for 10 days. The limbs are visually inspected for onset HFS 10 days later.

Treatment with Brimonidine

Skin tissue from the hind limbs is treated with a brimonidine topical formulation as described in any of the preceding examples. Treatment is measured by visual inspection or H&E, EdU or DAPI staining of skin tissue samples from the treatment area.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A method of treating a cell proliferation disorder in a subject in need thereof, comprising topically administering to a skin area of the subject a topical composition comprising an effective amount of at least one alpha adrenergic receptor agonist and a pharmaceutically acceptable carrier, wherein the skin area has, or is prone to have, the cell proliferation disorder; the alpha adrenergic receptor agonist is selected from the group consisting of (8-Bromo-quinoxalin-6-yl)-(4,5-dihydro-1H- imidazol-2-yl)-amine, (8-Bromo-quinoxalin-5-yl)-(4,5-dihydro-1H-imidazol-2-yl)-amine, (5-Bromo-3-methyl-quinoxalin-6-yl)-(4,5-dihydro-1H-imidazol-2-yl)-amine, (5-Bromo-2-methoxy-quinoxalin-6-yl)-(4,5-dihydro-1H-imidazol-2-yl)-amine, (4,5-dihydro-1H-imidazol-2-yl)-(8-methyl-quinoxalin-6-yl)-amine, (4,5-dihydro-1H-imidazol-2-yl)-quinoxalin-5-yl-amine, naphazoline, tetrahydrozoline, epinephrine, norepinephrine, phenylephrine, methoxamine, mephentermine, metaraminol, and midodrine; and the cell proliferation disorder is associated with one or more conditions selected from the group consisting of sun exposure, hormonal imbalance, a deficiency in vitamin and/or antioxidant, epidermal hyperplasia, keratinocyte proliferation, EGFR-dependent cell division, hand and foot syndrome, cutaneous T cell lymphoma, and combinations thereof.

2. The method of claim 1, wherein the cell proliferation disorder is the hand and foot syndrome.

3. The method of claim 2, wherein the hand and foot syndrome is induced by a chemotherapy.

4. The method of claim 1, wherein the cell proliferation disorder is cutaneous T cell lymphoma.

5. The method of claim 1, wherein the topical composition is selected from the group consisting of an aqueous solution topical formulation, a topical gel formulation, a cream topical formulation, and an ointment formulation.

6. The method of claim 1, wherein the cell proliferation disorder is further associated with one or more conditions selected from the group consisting of CREST syndrome, corns and calluses, warts, hives, keratosis, atopic dermatitis, eczema, scleroderma, lipoderamtoscelerosis, age spots (or lentigo).

7. The method of claim 1 wherein the cell proliferation disorder is induced by an UV-irradiation.

8. The method of claim 1, wherein the cell proliferation disorder is induced by a chemotherapy.

9. The method according to claim 1, wherein the composition comprises from about 0.01% to about 5% by weight of the alpha adrenergic receptor agonist.

10. The method according to claim 9, wherein the composition comprises from about 0.1%, 0.2%, 0.3%, 0.4%, 0.5%, 0.6%, 0.7%, 0.8%, 0.9% or 1.0% by weight of the alpha adrenergic receptor agonist.

11. The method of claim 10, wherein the alpha adrenergic receptor agonist is brimonidine.

12. The method of claim 1, further comprising administering to the subject at least one additional agent useful for reducing or inhibiting the progression of cell proliferation disorder.

13. The method of claim 12, wherein the additional agent is selected from the group consisting of retinoid and derivatives thereof, sun screens, sun-blocks, anti-inflammatory agents, vitamins and nitroglycerin.

14. The method of claim 12, wherein the additional agent and the at least one alpha adrenergic receptor agonist are administered to the subject in the same topical composition.

15. The method of claim 12, wherein the additional agent and the at least one alpha adrenergic receptor agonist are administered to the subject in separate topical compositions.

16. A method of treating hand and foot syndrome induced by chemotherapy in a subject in need thereof, comprising topically administering to a skin area of the subject a topical composition comprising an effective amount of brimonidine and a pharmaceutically acceptable carrier, wherein the skin area has, or is prone to have, the hand and foot syndrome.

17. The method of claim 16, further comprising administering to the subject at least one additional agent selected from the group consisting of retinoid and derivatives thereof, sun screens, sun-blocks, anti-inflammatory agents, vitamins and nitroglycerin.

18. A method of treating cutaneous T cell lymphoma in a subject in need thereof, comprising topically administering to a skin area of the subject a topical composition comprising an effective amount of brimonidine and a pharmaceutically acceptable carrier, wherein the skin area has, or is prone to have, the cutaneous T cell lymphoma.

19. The method of claim 18, further comprising administering to the subject at least one additional therapeutic agent useful for treating the cutaneous T cell lymphoma.

20. The method of claim 19, wherein the brimonidine and the additional therapeutic agent are administered to the subject simultaneously in the same topical composition, or separately.
